(19) United States
(12) Patent Application Publication
(10) Pub. No.: US 2015/0073423 A1
(43) Pub. Date: Mar. 12, 2015

(54) MEDICAL INSTRUMENT

(71) Applicant: Aesculap AG, Tuttlingen (DE)

(72) Inventors: Fabian Hoefer, Tuttlingen (DE); Sven Krueger, Trossingen (DE)

(21) Appl. No.: 14/456,181

(22) Filed: Aug. 11, 2014

(30) Foreign Application Priority Data
Sep. 10, 2013 (DE) 10 2013 109 895

(51) Int. Cl. A61B 17/88

(52) U.S. Cl. CPC A61B 17/8816; A61B 17/8811; A61B 17/8822; A61B 17/8875
USPC 606/94

(57) ABSTRACT

The present invention relates to a medical instrument which comprises a tool portion with a first tool element for engagement with a corresponding second tool element on an implant, and which further comprises a bone cement container arranged or formed on or in the instrument.

### CROSS-REFERENCE TO RELATED APPLICATIONS

This application claims the benefit of German application number 10 2013 109 895.9 filed on Sep. 10, 2013, which is incorporated herein by reference in its entirety and for all purposes.

### FIELD OF THE INVENTION

The present invention relates to medical instruments generally, and more specifically to a medical instrument comprising a tool portion with a first tool element for engagement with a corresponding second tool element on an implant.

### BACKGROUND OF THE INVENTION

Medical instruments of the above-described type are known in many and varied forms. For example, medical instruments are used which take the form of screwdrivers, in order to screw screws, in particular pedicle screws, of spine stabilisation systems into vertebrae of a human or animal spine. It is known to construct such screws with a cement channel, i.e. in particular in cannulated form, such that after introduction of the screws into a bone or bone part, for example into a pedicle of a vertebra, bone cement may additionally also be applied through the cement channel of the screw after implantation of the screw, in order to achieve even better anchoring of the bone screw.

### SUMMARY OF THE INVENTION

In a first aspect of the invention, a medical instrument comprises a tool portion with a first tool element for engagement with a corresponding second tool element on an implant. The medical instrument further comprises a bone cement container arranged or formed on or in the instrument.

### BRIEF DESCRIPTION OF THE DRAWINGS

The following description of preferred embodiments of the invention serves, in conjunction with the drawings, to provide a more detailed explanation. In the drawings:

FIG. 1: is a schematic perspective overall view of a medical instrument for introducing an implant into a bone part;

FIG. 2: is a longitudinal sectional view of a portion of a medical instrument coupled to an implant after introduction of the implant into the bone part;

FIG. 3: is a sectional view of a medical instrument with a first variant of a bone cement container; and

FIG. 4: is a sectional view similar to FIG. 3 of a further exemplary embodiment of a medical instrument with a mixing region.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a medical instrument comprising a tool portion with a first tool element for engagement with a corresponding second tool element on an implant, further comprising a bone cement container arranged or formed on or in the instrument.

The bone cement container allows bone cement, any desired fluid, a powder and/or an augmentation material optionally to be stored in the instrument itself. This makes it possible initially to introduce an implant with the instrument, and to do this by coupling the first tool element of the instrument with the corresponding second tool element of the implant. In the case, for example, of an implant in the form of a bone screw, the bone screw may in this way be screwed into a bone part, for example a vertebra. If the implant has reached its final position, it is then possible in a second step optionally to apply bone cement directly using the instrument, without having to remove the instrument again from the implant, as would be the case with conventional medical instruments. The bone cement is applied conventionally in a third step after removal of the screwing-in instrument from, and coupling for example of a cannula with bone cement to, the implant and then application of the bone cement through the implant. The medical instrument further developed according to the invention in this way in particular makes it markedly simpler to handle the bone cement. It is for example possible in this way to charge the instrument provided for introducing the implant with exactly the right amount of bone cement needed to cement the implant. In addition, external mixing of the bone cement is no longer necessary, such that soiling and contamination of the wound region can be avoided. In addition, soiling of the implant in the coupling region can also be avoided. If the instrument takes the form of a disposable instrument, simple disposal of the instrument and of the bone cement container is additionally possible. As already mentioned, the instrument may also be or have been filled with fluids, in particular rinsing fluids, or powders, and simplify application of the material contained in the bone cement container, since an implant can be introduced and then, without a change of instrument, the contents of the bone cement container can be applied.

The instrument may be of a particularly simple and compact construction if it comprises at least one portion in the form of a sleeve, and if the bone cement container is arranged or formed in the region of the portion in the form of a sleeve. Bone cement can thus be accommodated directly in the portion in the form of a sleeve. The wall thickness of the portion in the form of a sleeve is preferably selected so as to ensure that the instrument is sufficiently stable for introduction of the implant.

The portion in the form of a sleeve preferably forms or substantially forms the bone cement container. In this way, no additional elements are needed on the instrument to accommodate the bone cement. The instrument may for example be provided with a shank in the form of a sleeve instead of with a solid shank, bone cement being stored for example directly in the shank in the form of a sleeve or in a sleeve or sheath inserted therein.

According to a preferred embodiment of the invention, provision may be made for the bone cement container to comprise at least one first compartment and at least one second compartment and for the at least one first and the at least one second compartment to be separated from one another spatially by at least one separator element. The bone cement container may for example comprise two, three or four compartments. In particular for bone cement formed by mixing two components, the bone cement container preferably comprises two compartments. As a result of the at least one separator element, it is thus possible for the bone cement components contained in the compartments of the bone cement container to come into contact with one another only when this is desired. To this end, the separator element may for example be destroyed or removed, to deliberately allow mixing of the components of the bone cement.

It is favourable for the at least one separator element to take the form of a partition, which extends transversely of or parallel to a longitudinal axis of the bone cement container. For example, two compartments of the bone cement container may be arranged one behind the other in the longitudinal direction and be separated by a separator element extending transversely of the longitudinal axis. Alternatively, it is also feasible for two or more compartments to extend in the longitudinal direction of the bone cement container, wherein these may then be separated from one another by partitions likewise extending in the longitudinal direction. Alternatively, it is also feasible to accommodate the components of the bone cement in separate receptacles in the bone cement container, for example in thin-walled film packages. These may then for example be pierced, to enable mixing of the components. They may in particular be combined from the separate receptacles in a mixing chamber or mixing region.

It is advantageous for the instrument to comprise a feed device for intermixing and/or discharge of bone cement located in the bone cement container. In particular, the feed device may be constructed to feed the bone cement in the distal direction. The feed device allows bone cement to be applied simply and reliably through a cement channel of the implant.

The structure of the feed device is particularly simple if it comprises a plunger with a feed member conformed to an internal cross-section of the bone cement container. For example, the feed member may take the form of a small piston, which optionally comprises an encircling sealing element, in order to force the bone cement out of the preferably hollow-cylindrical bone cement container. In addition, the plunger may also be used for more sensitive force transmission and thus for more precise application of the contents of the bone cement container, for example bone cement or a fluid.

To be able to handle the instrument particularly simply, it is advantageous for the feed device to comprise an actuating element which defines a proximal end of the feed device and is temporarily coupled to the plunger or formed in one piece therewith. For example, the actuating element may project out of the instrument at the proximal end, such that a force may be exerted on it in the distal direction in order to force bone cement from the bone cement container out of the instrument in the distal direction.

The first tool element preferably comprises a bone cement channel, which passes through the first tool element and is in fluid connection with the bone cement container. It is thus possible to force bone cement directly through the first tool element into the implant, in particular even when the instrument is coupled with its first tool element to the implant.

It is favourable for the instrument to comprise a closing element which closes the bone cement channel and/or the bone cement container relative to the bone cement channel. The closing element thus in particular prevents bone cement from being able to escape through the bone cement channel. The closing element may also close the bone cement container relative to a mixing region or chamber.

It is favourable for the closing element to be irreversibly detachable or severable to open up the bone cement channel or the bone cement container. The bone cement accommodated in the bone cement container and/or the separately stored components thereof may in this way be discharged through the bone cement channel after detachment or severing of the closing element.

Bone cement may be introduced particularly deliberately and cleanly into the implant if the instrument comprises a cement outlet nozzle. This may preferably be formed to protrude beyond the first tool element at the distal end. In this way, bone cement can be prevented from exiting in the region of the first tool element and soiling the latter.

It is advantageous for the cement channel to extend through the cement outlet nozzle. In this way, bone cement may be introduced simply in the distal direction from the instrument into the implant.

According to a further preferred embodiment of the medical instrument, provision may be made for the bone cement container to comprise at least one mixing region, which comprises at least one mixing element and preferably a plurality of mixing elements. In the mixing region, which may in particular take the form of a mixing chamber, bone cement formed of two or more components may be mixed. For example two-component bone cement, which comprises a binder and a curing agent, may be optimally mixed in the mixing region in order to be able to start the desired chemical reaction between the components of the bone cement.

The mixing of two- or multi-component bone cement may be simply improved in that the plurality of mixing elements take the form of mixing projections, which are formed to project from an inner wall surface of the bone cement container. For example, these may take the form of vanes, such that on feed of the bone cement the latter does not flow in a laminar manner in the bone cement container but rather in a quasi-turbulent manner, so significantly improving mixing of the components of the bone cement.

It is advantageous for the bone cement container to contain bone cement. In other words, the instrument is then not only ready to accommodate bone cement in the bone cement container, but rather already contains bone cement, which can be applied directly out of the bone cement container after introduction of the implant without having to remove the instrument and then couple the implant to a bone cement applicator. The instrument in this way thus simultaneously also forms the cement applicator.

It is favourable for the bone cement to comprise at least one first bone cement component and at least one second bone cement component, which are contained in spatially separated manner in the bone cement container. This makes it possible also to store the bone cement in the instrument for a relatively long time, without the curing reaction being able to begin before actual application of the bone cement.

The at least one first compartment preferably contains the at least one first bone cement component and the at least one second compartment preferably contains the at least one second bone cement component. The bone cement components may thus be stored simply and reliably in a separated manner in the compartments of the bone cement container provided for this purpose, without their being able to react together in an undesired manner prior to application of the bone cement.

For simple and reliable handling of the instrument, it is favourable for it to comprise a handle element, which forms a proximal end of the instrument or is arranged or formed in the region of a proximal end of the instrument. In this way, it may be coupled simply and reliably, in particular with its distal end, to an implant, in order to introduce the latter directly into a bone part, and directly thereafter to apply bone cement.

The instrument may be of particularly compact construction if the handle element completely or partially comprises the bone cement container. Bone cement may thus be accommodated virtually inside the handle element.

In order to be able to partially reuse the instrument, it is advantageous for the handle element and bone cement container to be detachably connectable. Once implantation of the implant is complete and the bone cement has been applied, the handle element may thus for example be removed from the bone cement container, cleaned for the next implantation and then re-connected to a new bone cement container.

According to a further preferred embodiment of the invention, provision may be made for the first tool element to take the form of a tool projection for engagement with a correspondingly constructed tool receptacle on an implant. The instrument may thus be constructed to transmit a torque to the implant and be coupled simply and reliably to the implant.

Torques may be simply and reliably transmitted to an implant if the first tool element takes the form of a polygon, a multiple lobe member or a threaded portion. In this way, the instrument may be inserted or screwed in particular into a corresponding tool receptacle.

The instrument may be cheaply and simply produced if it is formed completely or partially from one or more plastics. It may for example be produced by injection moulding. In particular, different plastics may be used to form the first tool element and the bone cement container. To form the first tool element, plastics may for example be used which are only slightly deformable, i.e. are relatively hard. For the bone cement container, transparent or at least translucent plastics may preferably be used, so that an operator may directly perceive how much bone cement remains in the bone cement container.

The instrument may be produced particularly simply if it is formed entirely in one piece. It may for example be injection-moulded.

To screw in an implant, in particular a bone screw, it is advantageous for the instrument to take the form of a screwdriver.

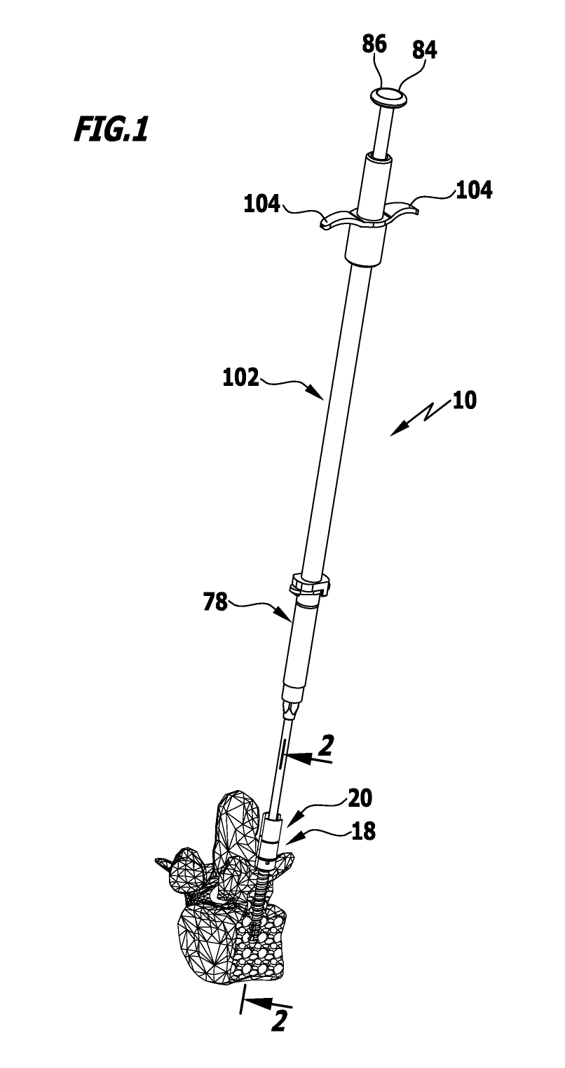

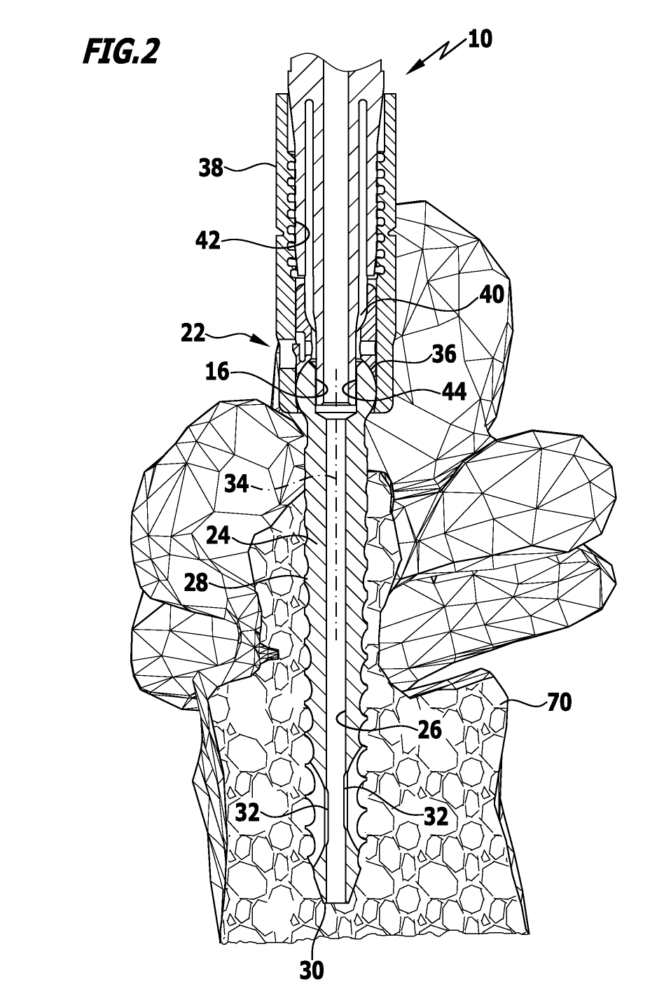

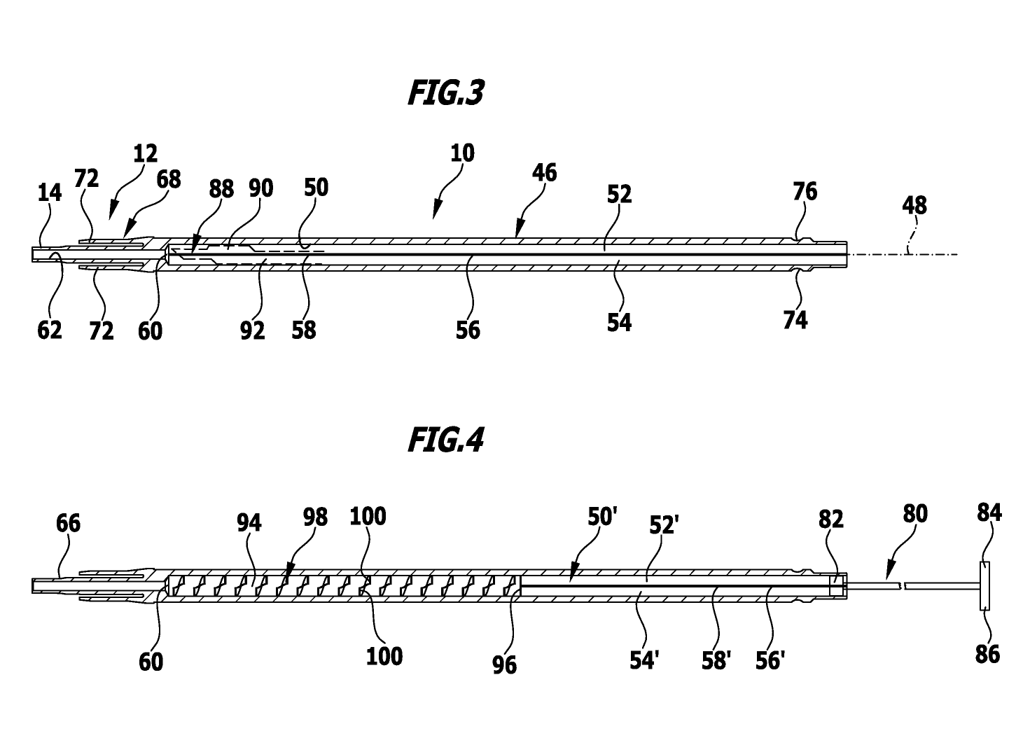

FIG. 1 is a schematic representation of a medical instrument designated with reference numeral 10. It comprises a tool portion 12 with a first tool element 66 for engagement with a second tool element 16 of an implant 18. The implant 18 may in particular take the form of a bone screw 20. In FIGS. 1 and 2 a bone screw 20 in the form of a polyaxial screw 22 is illustrated schematically by way of example, said polyaxial screw comprising a screw shank 24 through which passes a longitudinal channel 26 and which is provided with an external thread 28. A distal end 30 of the screw shank 24 is open and is in fluid connection with the longitudinal channel 26. One or more lateral outlet orifices 32 may optionally be provided to the proximal side of the end 30, which orifices open up the shank transversely of its longitudinal axis 34. The outlet orifices 32 are in fluid connection with the longitudinal channel 26.

The proximal end of the screw shank 24 takes the form of a spherical head 36, on which a sleeve 38 is mounted in articulated manner. The sleeve 38 serves in particular to accommodate rod- or plate-shaped connecting elements, which may be inserted with rod-shaped portions into a connecting element receptacle 40 of the sleeve 38. An internal thread 42 is formed on the sleeve 38, into which a fixing element, not shown in the Figures but taking the form of a screw, may be screwed to fasten the connecting element in the connecting element receptacle 40.

The head 36 has a tool receptacle 44 open in the proximal direction, which takes the form of a polygon socket or a multiple lobe socket. The tool receptacle 44 may optionally further be provided with an internal thread. Such a tool receptacle 44 is described for example in DE 20 2008 006 870 U1.

The instrument 10 comprises a portion 46 in the form of a sleeve, which defines a longitudinal axis 48. Inside the portion 46 a bone cement container 50 is arranged or formed, which comprises a first compartment 52 and a second compartment 54. These both extend in the direction of the longitudinal axis 48 and are separated by a separator element 56 in the form of a partition 58, which likewise extends in the direction of the longitudinal axis 48. At the distal end the hollow-cylindrical bone cement container 50 is closed relative to a bone cement channel 62 by a closing element 60.

The bone cement channel 62 passes through a cement outlet nozzle 64, which adjoins the distal end of the bone cement container 50 and extends coaxially relative to the longitudinal axis 48 in the distal direction. A distal end of the cement outlet nozzle 64 forms the first tool element 66, which may optionally take the form of an external polygon or external multiple lobe member or be provided with an external thread. The first tool element 66 projects beyond a coupling element 16. This optionally takes the form of a retaining clip 68, to couple the instrument 10 to the sleeve 38 in clamped manner in the region of said sleeve. The retaining clip 68 has two or more radially resilient projections 72 pointing in the distal direction and partially surrounding the cement outlet nozzle 64.

The first tool element 66 may either be screwed into the internal thread of the tool element receptacle 44 or, if it takes the form of an external polygon or external multiple lobe member, be inserted thereinto. The instrument 10 serves to transmit a torque to the head 36, such that the bone screw 20 may be screwed into a bone 70, for example a pedicle of a vertebra.

At a proximal end of the portion 46 an annular constriction 74 is formed, which defines an annular projection 76 at the distal end. The constriction 74 and projection 76 serve to couple the instrument 10 to an optional feed device 78. The feed device 78 serves in particular for intermixing and/or discharge of bone cement 88 located in the bone cement container 50. In particular, the feed device 78 serves to feed the bone cement 88 in the distal direction.

The feed device 78 in particular comprises a plunger 80 with a feed member 82 in the form of a piston, which forms a distal end of the plunger 80. A proximal end of the plunger 80 forms a flat, disc-like head 84, which serves as an actuating element 86 for the feed device 78. It is preferably formed nondetachably, in particular in one piece with the plunger 80.

Bone cement 88 is preferably already contained in the bone cement container 50, specifically a first component 90 in the first compartment 52 and a second component 92 in the second compartment 54. The two components 90 and 92, for example a binder and a curing agent, come into contact with one another and may react together if the separator element 56 is removed or destroyed.

To discharge the bone cement 88 using the feed device 78, the closing element 60 must be removed or destroyed. This may in particular be achieved by sufficient pressure being exerted on the bone cement 88 with the feed device 78.

The particular embodiment of the instrument 10 makes it possible to apply the bone cement 88 directly after the bone screw 20 has been screwed into the bone 70, without having to uncouple the instrument 10 from the bone screw 20 and couple on a separate applicator. In this way it is possible to prevent bone cement from exiting in the region of the sleeve 38 and soiling it, in particular in the region of the internal thread 42 and connecting element receptacle 40.

FIG. 4 shows by way of example a further exemplary embodiment of an instrument 10 with an alternative variant of the bone cement container 50′. The latter is somewhat shorter than the bone cement container 50. Thus, the portion 46 comprises not only the bone cement container 50′, but also a mixing chamber 94, which was also designated above as the mixing region. The latter extends between the bone cement channel 62 and the bone cement container 50′, which is separated from the mixing chamber 94 by a further closing element 96 extending transversely of the longitudinal axis 48. The separator element 56′ takes the form of a partition 58′ extending in the longitudinal direction and separates the first compartment 52′ from the second compartment 54′.

The instrument 10 further comprises a mixing device 98, which is preferably arranged in the mixing chamber 94. It comprises a plurality of mixing elements 100, which take the form of mixing projections which project towards the longitudinal axis 48. They are inclined somewhat relative to the longitudinal axis 48 and, subsequent to a feed movement of the plunger 80, lead to a to deflection of the bone cement 88 moving in the distal direction and to improved intermixing of the two components 90 and 92 in the mixing chamber 94. Through the mixing device 98 and the mixing chamber 94 optimum mixing of the two components 90 and 92 of the bone cement 88 and thus a defined curing reaction may be ensured.

Both of the instruments described may additionally comprise a handle element 102. This may in particular take the form of a conventional cannula and comprise two radially protruding vanes 104 at its proximal end, which an operator may rest his or her index finger and middle finger against while advancing the actuating element 86 in the distal direction with his or her thumb and applying the bone cement 88 or forcing it through the mixing chamber 94 prior to application.

The instrument 10 is preferably formed in one piece from sterilisable plastics. The instrument 10 may optionally also be made from a metal.

The instrument 10 makes it possible to combine the functions of a screwdriver, an applicator, a mixer and a reservoir for the bone cement 88 or the components 90 and 92 thereof. In this way, handling of the bone cement 88 may be simplified and at the same time the risk of undesired bone cement escape may be prevented.

Use of the instrument 10 and implantation of the implant 18 may proceed for example as follows:

First of all, the instrument 10 is prepared in sterile manner. The bone screw 20 is coupled with the instrument 10 and then placed on the bone 70 and screwed in.

In a next step the bone cement 88 is mixed, for example by breaking the separator element 56 or by pressing the two components 90 and 92 into the mixing chamber 94. The next step is to wait for the bone cement 88 to reach the viscosity needed for processing. As soon as it has reached this, it is applied with the plunger 80 through the bone cement channel 62 into the longitudinal channel 26.

One can now wait for the curing period. As soon as the bone cement 88 is sufficiently hard, the last step is to remove the instrument 10, for example by pulling or screwing it out, depending on how the instrument 10 was coupled to the bone screw 20.

As has already been mentioned, the coupling between the instrument 10 and the implant 18 preferably takes a form described in DE 20 2008 006 870. In other words, the instrument 10 is preferably coupled in cement-tight but air-permeable manner to the implant 18. This makes it possible to vent the instrument 10 in its function as applicator, so as to ensure that an excessive amount of air is not forced into the bone 70.

The size of the bone cement container 50 is preferably conformed to the implant 18 to be implanted with the instrument, such that exactly the correct amount of bone cement 88 is provided for the implant 18. With the instrument 10, external mixing is no longer necessary, such that there is no risk either of soiling or contamination of the wound region or of soiling of the bone screw 20.

The instrument 10 may be easily disposed of after use. It therefore preferably takes the form of a disposable instrument.

What is claimed is:

1. A medical instrument comprising a tool portion with a first tool element for engagement with a corresponding second tool element on an implant, further comprising a bone cement container arranged or formed on or in the instrument.

2. A medical instrument according to claim 1, further comprising at least one portion in the form of a sleeve, wherein the bone cement container is arranged or formed in the region of the portion in the form of a sleeve.

3. A medical instrument according to claim 2, wherein the portion in the form of a sleeve forms or substantially forms the bone cement container.

4. A medical instrument according to claim 1, wherein the bone cement container comprises at least one first compartment and at least one second compartment and wherein the at least one first and the at least one second compartment are separated from one another spatially by at least one separator element.

5. A medical instrument according to claim 4, wherein the at least one separator element takes the form of a partition, which extends transversely of or parallel to a longitudinal axis of the bone cement container.

6. A medical instrument according to claim 1, further comprising a feed device for intermixing and/or discharge of bone cement located in the bone cement container, in particular for feeding the bone cement in the distal direction.

7. A medical instrument according to claim 6, wherein the feed device comprises a plunger with a feed member conformed to an internal cross-section of the bone cement container.

8. A medical instrument according to claim 7, wherein the feed device comprises an actuating element which defines a proximal end of the feed device and is temporarily coupled to the plunger or formed in one piece therewith.

9. A medical instrument according to claim 1, wherein the first tool element comprises a bone cement channel, which passes through the first tool element and is in fluid connection with the bone cement container.

10. A medical instrument according to claim 9, further comprising a closing element, which closes the bone cement channel and/or the bone cement container relative to the bone cement channel.

11. A medical instrument according to claim 10, wherein the closing element is irreversibly detachable or severable to open up the bone cement channel.

12. A medical instrument according to claim 1, further comprising a cement outlet nozzle, which is formed to protrude beyond the first tool element at the distal end.

13. A medical instrument according to claim 12, wherein the bone cement channel extends through the cement outlet nozzle.

14. A medical instrument according to claim 1, wherein the bone cement container comprises at least one mixing region, which comprises a plurality of mixing elements.

15. A medical instrument according to claim 14, wherein the plurality of mixing elements take the form of mixing projections, which are formed to project from an inner wall surface of the mixing region.

16. A medical instrument according to claim 1, wherein the bone cement container contains bone cement.

17. A medical instrument according to claim 16, wherein the bone cement comprises at least one first bone cement component and one second bone cement component, which are contained in spatially separated manner in the bone cement container.

18. A medical instrument according to claim 17, wherein the at least one first compartment contains the at least one first bone cement component and wherein the at least one second compartment contains the at least one second bone cement component.

19. A medical instrument according to claim 1, further comprising a handle element, which forms a proximal end of the instrument or is arranged or formed in the region of a proximal end of the instrument.

20. A medical instrument according to claim 19, wherein the handle element completely or partially comprises the bone cement container or wherein the handle element and the bone cement container are connectable detachably.

21. A medical instrument according to claim 1, wherein the first tool element takes the form of a tool projection for engagement with a correspondingly formed tool receptacle on an implant.

22. A medical instrument according to claim 1, wherein the first tool element takes the form of a polygon, a multiple lobe member or a threaded portion.

23. A medical instrument according to claim 1, wherein the instrument is formed completely or partially from one or more plastics.

24. A medical instrument according to claim 1, wherein the instrument is formed entirely in one piece, in particular by injection moulding.

25. A medical instrument according to claim 1, wherein the instrument takes the form of a screwdriver.
